(19) United States
(12) Patent Application Publication
(10) Pub. No.: US 2013/0216539 A1
(43) Pub. Date: Aug. 22, 2013

(54) Methods of Generation and Treatment with Modified Derivatives of HSP 70

(71) Applicant: ALS Biopharma, LLC, (US)

(72) Inventors: Allen B. Reitz, Lansdale, PA (US); Aaron Pawlyk, Silver Spring, MD (US); Sergey Ilyin, Oreland, PA (US)

(73) Assignee: ALS Biopharma, LLC, Doylestown, PA (US)

(21) Appl. No.: 13/768,242

(22) Filed: Feb. 15, 2013

Related U.S. Application Data
(60) Provisional application No. 61/599,632, filed on Feb. 16, 2012.

(51) Int. Cl. A61K 47/48; C07K 14/47

(52) U.S. Cl. CPC A61K 47/48215; C07K 14/47; A61K 47/48415
USPC 424/134.1; 530/410; 435/69.6; 530/387.3; 435/328; 435/375; 514/1.1; 514/15.1; 514/1.8; 514/1.4; 514/19.2; 514/13.5; 514/18.2; 514/20.8; 514/17.7; 514/15.4; 514/18.6

(57) ABSTRACT

The present invention includes methods of generating derivatives of a protein, as well as methods of treating a subject with the derivatized proteins. More particularly, the present invention includes methods of generating derivatives of HSP 70 proteins and methods of treating a subject with the derivatized HSP 70 proteins.

### CROSS-REFERENCE TO RELATED APPLICATIONS

The present application claims priority to U.S. Provisional Application No. 61/599,632, filed Feb. 16, 2012, which application is hereby incorporated by reference in its entirety herein.

### BACKGROUND OF THE INVENTION

Heat shock proteins (HSPs) are known to protect cells, tissues, and subjects from a variety of stressors, such as heat shock stress, oxidative stress, protein misfolding stress, and glutamate excitotoxicity. The HSP 70 proteins are the most abundant cellular HSPs and include constitutively expressed forms (HSC 70, also referred to as HSP 70) as well as inducible forms (HSP 72, also referred to as HSP 70), all of which are collectively as HSP 70 herein. Induction of HSP within cells has been shown to decrease cellular stress and prevent premature cell death. While many proteins are capable of spontaneously refolding or, when misfolded, are degraded by the cellular machinery, other proteins require active refolding when misfolded to either regain function or promote clearance.

It is also now recognized that cells can secrete HSP 70 into the extracellular environment and thus exert an effect on additional cell types via this mechanism. The effect of extracellular HSP 70 is varied and may be linked to multiple cellular mechanisms. It has been shown that cells, specifically neurons, can take up HSP 70 from the surrounding media. It has also been shown that HSP 70 can bind to cell surface receptors, exerting cellular and physiologic effects.

Treatment with native proteins can be problematic due to the poor pharmacokinetics and the cost of manufacture of such proteins. Most enzymes and proteins administered exogenously to animals display poor PK properties and are rapidly cleared from circulation. While the resulting poor exposure can be overcome by frequent administration of high doses of a therapeutic protein, this may require frequent dosing with a large volume of solution, raising issues of patient tolerability. Additionally, manufacturing costs for biologic therapeutics are much greater than for small molecule therapeutics and large-dose administration of biological therapeutics may become cost prohibitive.

Numerous approaches have been put forward for the modification of proteins in order to improve pharmacokinetics and/or pharmacodynamics as essential to treatment and/or reduce the quantity necessary for treatment as a means of reducing production costs. For large proteins and enzymes, a process known as PEGylation has been found to be highly effective. In this approach, polyethylene glycol (PEG or peg) chains [—(OCH₂CH₂)ₙ—] of defined and homogeneous composition are covalently attached onto one or more amino acids, such as the N-terminal amino group. The derivatization reaction is carefully selected to preserve biological activity while enhancing half-life and reducing immunogenicity of the protein. Importantly, PEGylation has been successfully used in several FDA-approved clinical products such as Mircera® (peg-EPO), Doxil/Caelyx®, Pegasys®, Pegintron®, Oncaspar®, and Neulasta®. Mircera®, for example, required only once-monthly treatment for renal anaemia in patients with chronic kidney disease (CKD) and renal failure.

Despite the interest in the heat shock proteins (HSPs) and particularly HSP 70 (see U.S. Pat. No. 5,348,945), to date no specific HSP 70-based product has been brought to market. There is need in the art for a derivative of HSP 70 that displays improved pharmacokinetics without detrimental modification of its biological activity. This invention addresses this need.

### BRIEF SUMMARY OF THE INVENTION

The invention includes a method of enhancing the activity of an isolated HSP70 molecule. The method comprises covalently bonding to the isolated HSP70 molecule at least one PEG molecule having an approximate weight average molecular weight in the range of 20,000 to 40,000.

The invention further includes a method of generating derivatives of an isolated HSP70 molecule. The method comprises PEGylating the isolated HSP70 molecule using reductive amination.

The invention further includes a method of generating derivatives of an isolated HSP70 molecule. The method comprises PEGylating the isolated HSP70 molecule using a reaction other than reductive amination.

The invention further includes a composition comprising a PEGylated HSP 70 molecule, wherein between one and ten PEG groups are attached to the molecule.

The invention further includes a method of generating a fusion of HSP 70 with the Fc fragment of an antibody.

The invention further includes a composition comprising a recombinant HSP 70:Fc fragment fusion protein comprising a leader peptide, HSP 70, a peptide linker, and a human IgG Fc variant, wherein the variant is comprised of a hinge, CH2, and CH3 domains of human IgG1. In one embodiment, the peptide linker comprises 20 or fewer amino acids and is present between the HSP 70 and the human IgG Fc variant; and wherein the peptide linker comprises two or more amino acids selected from the group consisting of glycine, serine, alanine, and threonine.

The invention further includes a composition comprising a CHO cell line transfected with DNA encoding a fusion protein of the invention, wherein the fusion protein is expressed in its growth medium in excess of 10 μg per million cells in a 24 hour period.

The invention further includes a composition comprising a recombinant fusion protein comprised of HSP 70, a flexible peptide linker, and a human IgG Fc variant, wherein the variant comprises a hinge, CH2, and CH3 domains of human IgG1. The fusion protein is generated by the method comprising the steps of: (a) generating a CHO cell line transfected with DNA encoding the recombinant fusion protein; (b) growing the cell line under conditions wherein the recombinant fusion protein is expressed in its growth medium in excess of 10 μg per million cells in a 24 hour period; and (c) purifying the expressed protein from step (b).

In one embodiment, the flexible peptide linker contains 20 or fewer amino acids and is present between the HSP 70 and the human IgG Fc variant; and wherein the peptide linker comprises two or more amino acids selected from the group consisting of glycine, serine, alanine, and threonine.

The invention further includes a method of enhancing the survivability of a cell, tissue, or subject under stress. The method comprises contacting exogenous HSP 70 derivatives with the cell, tissue, or subject in an amount effective to enhance the survivability of the cell, tissues, or subject.

In one embodiment, the cell is part of a primary neuron culture. In another embodiment, the cell is part of a primary motoneuron culture. In yet another embodiment, the subject is an animal model for a human disease state or process. In yet another embodiment, the state or process is selected from the group consisting of cerebral ischaemia, cystic fibrosis, myocardial infarction, inflammatory disorders, hepatotoxicity, sepsis, organ or tissue transplant rejection, tumorous diseases, gastric mucosal damage, brain hemorrhage, diabetic neuropathy, diabetic retinopathy, chronic wound healing associated with diabetes and other disorders, neurodegenerative diseases, amyotrophic lateral sclerosis, Parkinson's disease, frontotemporal lobar degeneration, epilepsy, post-traumatic neuronal damage, acute renal failure, glaucoma, skin degeneration, and celiac disorder. In yet another embodiment, the subject is a human patient. In yet another embodiment, the patient was diagnosed with a state or process selected from the group consisting of cerebral ischaemia, cystic fibrosis, myocardial infarction, inflammatory disorders, hepatotoxicity, sepsis, organ or tissue transplant rejection, tumorous diseases, gastric mucosal damage, brain hemorrhage, diabetic neuropathy, diabetic retinopathy, chronic wound healing associated with diabetes and other disorders, neurodegenerative diseases, amyotrophic lateral sclerosis, Parkinson's disease, frontotemporal lobar degeneration, epilepsy, post-traumatic neuronal damage, acute renal failure, glaucoma, skin degeneration, and celiac disorder.

### BRIEF DESCRIPTION OF THE FIGURES

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A-1B, illustrates fusion proteins of the invention. FIG. 1A is a schematic diagram illustrating the general structure of the recombinant human HSP70:Fc fusion protein according to a non-limiting embodiment of the present invention. FIG. 1B is a schematic diagram illustrating the general structure of the recombinant human HSP70:Fc fusion protein according to a non-limiting embodiment of the present invention.

FIG. 2, comprising FIGS. 2A-2B, illustrates fusion proteins of the invention. FIG. 2A is a schematic diagram illustrating the general structure of the recombinant human HSP70:Fc fusion protein according to a non-limiting embodiment of the present invention. FIG. 2B is a schematic diagram illustrating the general structure of the recombinant human HSP70:Fc fusion protein according to a non-limiting embodiment of the present invention.

FIG. 3 is a graph illustrating activity of native HSP70 (closed triangles), 20,000 MW PEG-HSP70 (closed squares) and 40,000 MW PEG-HSP70 (stars) in refolding assay.

FIG. 4 is a graph illustrating PK in mouse serum of native HSP70 (open squares), 20,000 MW PEG-HSP70 (triangles) and 40,000 MW PEG-HSP70 (circles) after bolus iv injection.

### DETAILED DESCRIPTION OF THE INVENTION

The present invention relates to the unexpected discovery that PEG-modified HSP70 derivatives prepared according to the methods of the present invention have a significantly improved PK over the unmodified HSP70. It was surprisingly discovered that PEG-40000 HSP70, prepared herein, has a significantly better PK than PEG-20000 HSP70.

The present invention also includes a construct comprising a leader sequence to facilitate secretion of HSP 70 or an HSP 70:Fc fragment fusion. The present invention also includes a method of generating an Fc fragment:HSP 70 fusion protein, and a method of enhancing the survivability of cells, tissues, and organisms by treating the same with an HSP 70 derivative.

### DEFINITIONS

It is advantageous to define several terms before describing the invention. It should be appreciated that the following definitions are used throughout this application.

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “isolated” means altered or removed from the natural state through the actions of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as a host cell for example.

As used herein, the term “HSP 70” refers to HSPs of approximately 70 kDa from a particular species or, in cases where HSP 70 denotes a family of closely related proteins all found in that species, the term refers to that family of proteins. The term “HSP 70” is intended to include the full-length HSP 70 or and fragments or subunits of any HSP 70, including those recombinantly or chemically modified, regardless of whether or not those fragments or subunits have activity prior to PEGylation or fusion with an Fc fragment.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks, directly or indirectly, the biological activity of the antigen.

As used herein, the term “monoclonal antibody” includes antibodies that display a single binding specificity and affinity for a particular epitope. These antibodies are mammalian-derived antibodies, including murine, human and humanized antibodies. As used herein, an “antibody heavy chain” refers to the larger of the two types of polypeptide chains present in all antibody molecules in their naturally occurring conformations. As used herein, an “antibody light chain” refers to the smaller of the two types of polypeptide chains present in all antibody molecules in their naturally occurring conformations.

As used herein, the term “Fc fragment” refers to the fragment crystallizable region (Fc region) that is the tail region of an antibody, and refers to any protein sequence from any species that has particular utility. Preferred forms would be a hinge, CH2 and CH3 domains of human IgG such as human IgG1, IgG2, and IgG4. As used herein, the term “derivative” refers to a small molecule that differs in structure from the reference molecule, but retains the essential properties of the reference molecule. A derivative molecule may also include a salt, an adduct, or other variant of the reference molecule.

As used herein, the term “HSP 70 derivatives” refers to either mono-PEGylated derivatives of HSP 70 or to the fusion of HSP 70 with and Fc fragment.

The term “clearance,” as used herein refers to the physiological process of removing a compound or molecule, such as by diffusion, exfoliation, removal via the bloodstream, and excretion in urine, or via other sweat or other fluid.

As used herein, the term “subject” or “patient” or “individual” refers to a human or a non-human mammal. Non-human mammals include, for example, livestock and pets, such as ovine, bovine, porcine, canine, feline and murine mammals. In one embodiment, the subject is canine, feline or human. In another embodiment, the subject is human.

As used herein, the term “effective amount” or “therapeutically effective amount” of a compound is that amount of compound which is sufficient to provide a beneficial effect to the subject to which the compound is administered.

As used herein, the term “pharmaceutically acceptable” refers to a material, such as a carrier or diluent, which does not abrogate the biological activity or properties of the compound useful within the invention, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

As used herein, the term “composition” or “pharmaceutical composition” refers to a mixture of at least one compound useful within the invention with a pharmaceutically acceptable carrier. The pharmaceutical composition facilitates administration of the compound to a subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “p”Pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the construct's ability to perform its intended function, e.g., treating, ameliorating, or preventing a disease or disorder in a subject.

As used herein, the term “applicator” is used to identify any device including, but not limited to, a hypodermic syringe, a pipette, and the like, for administering the compositions used in the practice of the invention.

As used herein, the term “instructional material” includes a publication, a recording, a diagram, a product insert or any other medium of expression that may be used to communicate the usefulness of the composition and/or compound of the invention in the kit with respect to the methods of the invention. Optionally, or alternately, the instructional material may describe one or more methods related to the present invention, including as disclosed elsewhere herein.

The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Alternately, the instructional material may be obtained on the Internet in a format suitable for electronic file transmission to the user. For example, the instructional material is for use of a kit; instructions for use of the compound; or instructions for use of a formulation of the compound.

### Compositions of the Invention

The invention includes a composition comprising a PEGylated HSP 70 molecule, wherein between one and ten PEG groups are attached to the molecule.

The invention also includes a composition comprising a recombinant HSP 70:Fc fragment fusion protein comprising a leader peptide, HSP 70, a peptide linker, and a human IgG Fc variant, wherein the variant is comprised of a hinge, CH2, and CH3 domains of human IgG1. In one embodiment, the peptide linker contains 20 or fewer amino acids and is present between the HSP 70 and the human IgG Fc variant; and the peptide linker comprises two or more amino acids selected from the group consisting of glycine, serine, alanine, and threonine.

The invention further includes a composition comprising a CHO cell line transfected with DNA encoding the fusion protein of the invention, wherein the fusion protein is expressed in its growth medium in excess of 10 μg per million cells in a 24 hour period.

The invention also includes a composition comprising a recombinant fusion protein comprised of HSP 70, a flexible peptide linker, and a human IgG Fc variant, wherein the variant comprises a hinge, CH2, and CH3 domains of human IgG1. The fusion protein is generated by the method comprising the steps of generating a CHO cell line transfected with DNA encoding the recombinant fusion protein; the method further comprises the step of growing the cell line under conditions wherein the recombinant fusion protein is expressed in its growth medium in excess of 10 μg per million cells in a 24 hour period; the method further comprises the step of purifying the expressed protein from the previous step.

In one embodiment, the flexible peptide linker contains 20 or fewer amino acids and is present between the HSP 70 and the human IgG Fc variant; and wherein the peptide linker comprises two or more amino acids selected from the group consisting of glycine, serine, alanine, and threonine.

In one aspect, the invention includes a construct comprised of a leader sequence to facilitate secretion of HSP 70 or an HSP 70-Fc fragment fusion.

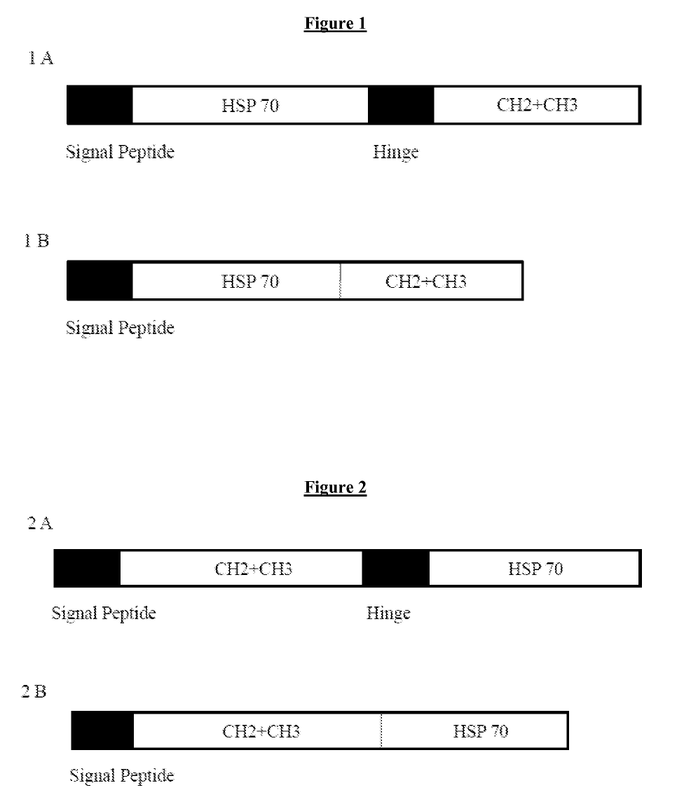

HSP 70 is an intracellular protein and the addition of a leader peptide to facilitate secretion may enhance production and purification of the recombinant protein as shown schematically in FIGS. 1A-1B and 2A-2B.

Suitable DNA encoding a leader peptide may be obtained by PCR cloning or de novo synthesis or other methods available to one skilled in the art of molecule biology. The DNA encoding a leader peptide sequence may be inserted at the 5′- or 3′-ends of either the DNA encoding the HSP 70 or Fc fragment. The sequence may be used to generate constructs with HSP 70 alone or with HSP 70: Fc fragment fusion constructs.

### Methods of the Invention

In one aspect, the present invention includes a method of generating a series of PEGylated HSP 70 derivatives.

In general, chemical derivatization may be performed by reacting a biologically active substance with an activated polymer molecules under a suitable assay condition. The method of preparing PEGylated HSP 70 comprises the step of reacting an HSP 70 with a reactive polyethylene glycol derivative, such as a reactive ester or aldehyde derivative, under conditions in which one or more PEG groups becomes covalently attached to HSP 70. The method further comprises the step of isolating the reaction products. As each protein behaves differently in response to PEGylation chemistry, the optimal conditions for PEGylation of HSP 70 have to be determined empirically by varying known parameters. As non-limiting examples, the smaller the ratio of HSP 70 to PEG is, the greater the poly-PEGylated product is. Attachment of PEG to HSP 70 is in general conducted by carrying out reductive amination conditions at a pH suitable to permit selective derivatization of the N-terminal amino group alone. Reaction products are then obtained by methods available to one skilled in the art.

Molecular weight and branching of the PEG polymer is also an important parameter in the PEG coupling reactions. In general, larger or branched PEG polymers require a greater PEG: HSP 70 ratio. In general, for the PEGylation reactions contemplated for generation of PEGylated derivatives, the preferred average molecular weight for the PEG derivative is 2±1 kDa to 100±1 kDa. For linear PEG changes the preferred average molecular weight for the PEG derivative is 10±1 kDa to 50±1 kDa, and for branched PEG polymers the preferred molecular weight is 20±1 kDa to 80±1 kDa. A ratio of PEG polymer to HSP 70 protein generally ranges from 1:1 to 100:1 and is preferably 1:1 to 30:1 for poly-PEGylation and from 1:1 to 1:10 for mono-PEGylation.

In another aspect, the present invention includes a method of generating an Fc fragment:HSP 70 fusion protein as shown schematically in FIGS. 1A-1B and 2A-2B.

The gene encoding a fusion protein may be assembled from several DNA segments. The gene encoding the Fc region of human IgG1 or IgG2 may be obtained by a method such reverse transcription and PCR using RNA prepared from human leukocytes and appropriate 5′ primers or to other methods obvious to one skilled in the art of molecular biology. Resulting DNA fragment encoding the Fc fragment are comprised of the hinge, CH2, and CH3 domains of IgG. The sequence of the gene may be confirmed by DNA sequencing.

In a non-limiting example, to prepare the HSP 70:Fc fusion gene, the HSP 70 fragment is excised from a plasmid containing the DNA sequence encoding for HSP 70 with appropriate restriction sites or restriction sites added by one skilled in the art and is then purified by agarose gel electrophoresis. The purified fragment is then inserted at either the 5′-end or 3′-end of the peptide linker in the Fc containing plasmid to give the plasmid containing the DNA sequence for the HSP 70:Fc fragment. The Fc fragment may be either on the N- or C-termini of the fusion protein. The fusion gene therefore comprises DNA encoding for HSP 70, a peptide linker such as Gly-Ser and the Fc fragment. Additional peptide linker sequences may be used by one skilled in the art.

The presence of a peptide linker between the HSP 70 and Fc moieties increases the flexibility of the HSP 70 domains and enhances its biological activity as shown schematically in FIG. 1A and FIG. 2A.

For the present invention, a peptide linker of about 20 or fewer amino acids in length is preferred. Peptide linker, comprising two or more of the following amino acids glycine, serine, alanine, and threonine, can be used. An example of the peptide linker contains Gly-Ser peptide building blocks, such as GlyGlyGlyGlySer. The IgG Fc variant is of non-lytic nature and contains amino acid mutations as compared to naturally occurring IgG Fc. This aspect of the invention may or may not contain a linker between the secretion sequence and rest of the fusion protein. A preferred form comprises a flexible peptide linker of 20 or fewer amino acids in length which comprises two or more of the following amino acids: glycine, serine, alanine, and threonine. The IgG Fc variant is of non-lytic nature and contains amino acid mutations as compared to naturally occurring IgG Fc.

The complete DNA sequence encoding the HSP 70:Fc fragment fusion protein may then be cloned into a vector suitable for expression in eukaryotic cells of origin from any number of animal, plant, or fungal species by one skilled in the art of molecular biology. The final expression vector contains a promoter appropriate for high level expression in the selected cell type as well as the sequence encoding the DNA for the HSP 70—Fc fragment fusion protein. The vector also contains genes expression antibiotic or other resistance for selective pressure during routine work and or during expression of the fusion construct.

In a preferred embodiment, a complete gene encoding the HSP 70:Fc fragment fusion protein inserted at the HindIII and EcoRI sites of a mammalian expression vector, such as pcDNA3.2 (Invitrogen) is provided. The final expression vector plasmid, contains the cytomegalovirus early gene promoter-enhancer which is required for high level expression in mammalian cells. The plasmid also contains selectable markers to confer ampicillin resistance in bacteria, and G418 resistance in mammalian cells.

In another aspect, the invention includes a method of enhancing the survivability of cells, tissues, and subjects by treating the same with an HSP 70 derivative. In one embodiment, neurons, either in isolation or in co-culture with additional cell types, from a species of interest are isolated from an animal and maintained in standard cell culture conditions. Cultured neurons are then subjected to a form of stress such as heat shock stress, oxidative stress, or excess levels of glutamate. HSP 70 derivates and vehicle are tested under these stressed conditions to assess the ability of the HSP 70 derivative to protect neurons and/or other cell types from stressors.

A preferred HSP 70 derivative increases the survival or health of neurons or other cell types as assessed by microscopic analysis, standard cell viability assays, or other methods available to one skilled in the art of assessing cell health.

The ability of an HSP 70 derivative to impact the health of non-neuronal cultures is also embodied by this invention and may be undertaken by one skilled in the art of those particular tissues types.

In accordance with an embodiment of the present invention, an HSP 70 derivative may be administered to an animal by any route in order to test its effect on disease progression in a variety of animal models. Animal models may be generated by knockout or knockdown, transgenic expression of wild type or mutated genes, selection from a normal pool of animals, surgical modification, pharmacological manipulation, behavioral or affective conditioning, dietary control, extrapolation of a measure to a disease state, or other methods available to one skilled in the art. An HSP 70 derivative is delivered in the same route as a vehicle control either acutely or as multiple doses across an appropriate time period for the animal model.

A preferred HSP 70 derivative improves one or more of the following compared to a vehicle control: overall survivability of the animals, delay in onset to symptoms, decrease in symptom severity, a recognized positive effect to a disease biomarker, or a recognized positive effect to a surrogate endpoint.

Based on the known mechanisms of action of HSP 70, PEGylated derivatives of HSP 70 and HSP 70:Fc fragment fusion proteins are expected to be effective in their own or in combination therapies in the treatment of the following diseases: cerebral ischaemia, cystic fibrosis, myocardial infarction, inflammatory disorders, hepatotoxicity, sepsis, organ or tissue transplant rejection, tumorous diseases, gastric mucosal damage, brain hemorrhage, diabetic neuropathy, diabetic retinopathy, chronic wound healing associated with diabetes are other disorders, neurodegenerative diseases such as Alzheimer's disease, amyotrophic lateral sclerosis, Parkinson's disease, frontotemporal lobar degeneration, epilepsy, post-traumatic neuronal damage, acute renal failure, glaucoma, skin degeneration, celiac disorder.

HSP 70 derivatives may be formulated in a variety of manner suitable for oral, rectal, topical, nasal, opthalamic or parenteral (including subcutaneous, intramuscular, intravenous, or intraperitoneal) administration. Additional routes of administration may be used for practicing the present invention such as intrathecal administration directly into cerebral spinal fluid, injection onto an arterial surface to reduce arteriosclerosis or to prevent re-stenosis of a stent, placement into at attached or embedded surface of a stent for the reduction of arteriosclerosis or to prevent re-stenosis of a stent, and intraparenchymal injection directly into targeted areas of an organ. The formulations may be prepared by any of the methods known to one skilled in the arts of pharmacy.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Activity of PEGylated HSP 70 Protein

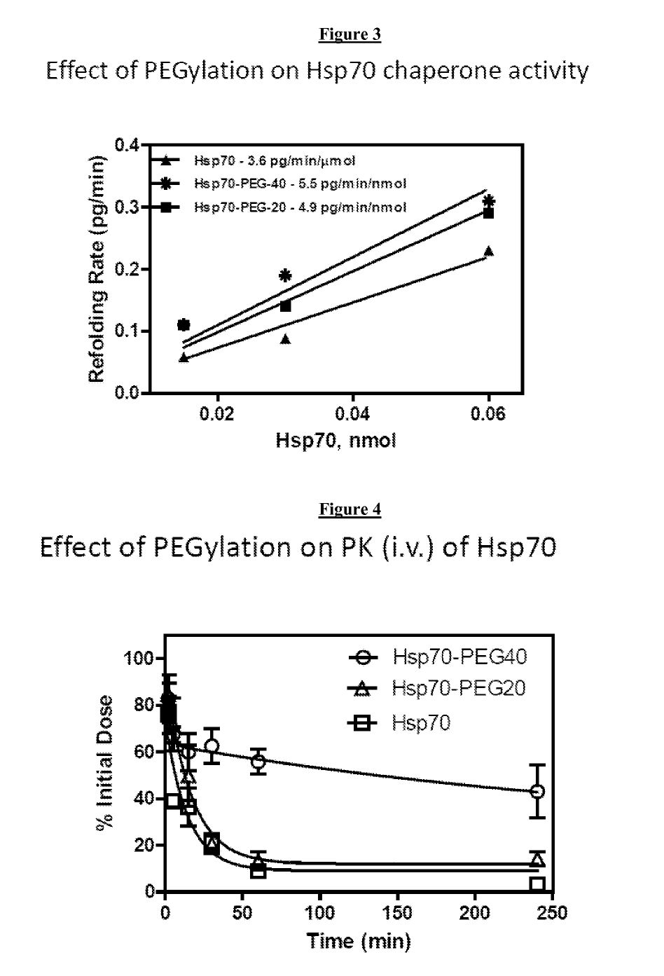

HSP 70's chaperone activity was determined by monitoring its ability to refold the enzyme luciferase essentially as described in Wisen et al. (Anal. Biochem. 2008, 374:371-7). Luciferase (0.5 mg/mL) was denatured in buffer A (25 mM HEPES, pH 7.4, 50 mM potassium acetate, 5 mM DTT) containing 6 M guanidine HC1 for 60 min at room temperature. The denatured luciferase was then diluted to 25 μg/mL in buffer A and stored at −80° C. in aliquots until use. Assays contained a range of concentrations of HSP 70, or derivative, in 45 μL of refolding buffer 28 mM HEPES, pH 7.4, 120 mM potassium acetate, 12 mM magnesium acetate, 2.2 mM DTT, 0.1 mM ATP, 8.8 mM creatine phosphate, 35 U/mL creatine phosphokinase, 48 nM DnaJ). At various times, 5 μL aliquots were removed and dispensed into 50 μL luciferase assay buffer (75 mM Tricine, pH 7.8, 24 mM MgSO4, 0.3 mM EDTA, 2 mM DTT, 160 μM D-luciferin, 320 μM coenzyme A, 0.66 mM ATP, 150 mM KCl, 20% glycerol, 10% Triton X100, 3.5% DMSO) as described by (Galam et al., Bioorg. Med. Chem. 2007 15:1939-46). Luminescence was measured using a TopCount scintillation counter (PerkinElmer, Waltham, Mass.). Rates of luciferase refolding at different concentration of Hsp70, or derivative, are determined by linear regression of the incubation times tested and then plotted against HSP 70 or derivative concentration to obtain specific activity as pg luciferase refolded per min per nanomole of HSP 70 as indicated in FIG. 3.

### Example 2

### Pharmacokinetics of HSP 70 Derivatives

PEG-HSP 70 or HSP70 was administered at a dose, such as 3 μg Hsp70-equivalent (i.e. for a 110 kDa PEG-Hsp70, a dose of 4.7 μg would contain 3 μg Hsp70), to groups of 3-12 mice for each time point and dosing route. HSP70 ELISA. Concentrations of Hsp70 in plasma were determined by ELISA as illustrated in FIG. 4. Black opaque 96-well plates (MaxiSorp® Nalgene Nunc International, Rochester, N.Y.) were coated with 100 μL of 0.83 μg/mL mouse monoclonal anti-HSP 70 antibody (SPA-8136, Enzo Life Sciences, Plymouth Meeting, Pa.) in PBS overnight at 4° C. After coating, the wells were washed 3×150 μL of wash buffer (TBS+0.1% Tween 20) with shaking for 1-2 min between washes. Wells were then blocked with 100 μL of 5% nonfat dry milk in PBS for 30 min at room temperature. After blocking, the wells were rinsed with PBS and samples containing unknown amounts of HSP 70 (100 μL) are added to appropriate wells. In addition, a standard curve of HSP 70 (Enzo Life Sciences, Plymouth Meeting, Pa.) ranging from 200 ng/mL to 12.5 ng/mL is prepared in the same media as the unknown samples and 100 μL of each concentration is added to appropriate wells. The assays are then incubated for 2 hr at room temperature with gentle shaking After incubation, the samples are removed and the wells washed 3×150 μL wash buffer as before. Rabbit polyclonal anti-HSP 70 antibody (SPA-812, Enzo Life Sciences, Plymouth Meeting, Pa.) is diluted to 0.83 μg/mL in antibody buffer (PBS+0.25% BSA) and 100 μL added to each well followed by another 2 hr incubation at room temperature with shaking. After incubation the wells washed 4×150 μL wash buffer as before. Goat anti-rabbit HRP conjugated antibody (7074, Cell Signaling Technology, Danvers, Mass.) is diluted to 0.83 μg/mL in antibody buffer and 100 μL added to each well followed by a 1 hr incubation at room temperature with shaking. After incubation, the wells are washed 4×150 μL wash buffer as before and 100 μL of PBS, 2 mM H₂O₂, 20 μM Amplex Red® (Invitrogen, Carlsbad, Calif.) is added to each well. After a 30 min incubation at room temperature, fluorescence is measured at excitation λ 530 nm and emission λ 590 nm using a Synergy 2 plate reader (BioTek, Winooski, Vt.). Hsp70 concentrations are determined based on linear regression fit of the standard curve.

PEG-HSP70 ELISA. Concentrations of PEG-HSP 70 in plasma or other media was determined by ELISA. Black opaque 96-well plates (MaxiSorp® Nalgene Nunc International, Rochester, N.Y.) were coated with 100 μL of 2 μg/mL rabbit monoclonal anti-PEG antibody (2061-1, Epitomics, Burlingame, Calif.) in 50 mM NaHCO₃, pH 9.0 overnight at 4° C. After coating, the wells were washed 3×200 μL of wash buffer (TBS+0.02% CHAPS) with shaking for 1-2 min between washes. Wells were then blocked with 100 μL of 5% nonfat dry milk in PBS for 30 min at room temperature. After blocking, the wells were rinsed with PBS and samples containing unknown amounts of PEG HSP70 (100 μL) are added to appropriate wells. In addition, a standard curve of the appropriate PEG-HSP 70 derivative ranging from 200 ng/mL to 12.5 ng/mL is prepared in the same media as the unknown samples and 100 μL of each concentration is added to appropriate wells. The assays are then incubated for 2 hr at room temperature with gentle shaking. After incubation, the samples are removed and the wells washed 3×200 μL wash buffer as before. Rabbit polyclonal anti-HSP 70 biotin conjugated antibody (ab79648, Abcam, Cambridge, Mass.) is diluted to 0.53 μg/mL in antibody buffer (PBS+0.25% BSA) and 100 μL added to each well followed by another 2 hr incubation at room temperature with shaking. After incubation the wells washed 4×200 μL wash buffer as before. Strepavidin-HRP (3999, Cell Signaling Technology, Danvers, Mass.) is diluted to 0.50 μg/mL in antibody buffer and 100 μL added to each well followed by a 1 hr incubation at room temperature with shaking. After incubation, the wells are washed 4×200 μL wash buffer as before and 100 μL of PBS, 2 mM H₂O₂, 20 μM Amplex Red® (Invitrogen, Carlsbad, Calif.) is added to each well. After a 30 min incubation at room temperature, fluorescence is measured at excitation λ 530 nm and emission λ 590 nm using a Synergy 2 plate reader (BioTek, Winooski, Vt.). PEG-HSP 70 concentrations are determined based on linear regression fit of the standard curve.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of enhancing the activity of an isolated HSP70 molecule, the method comprising covalently bonding to the isolated HSP70 molecule at least one PEG molecule having an approximate weight average molecular weight in the range of 20,000 to 40,000.

2. A method of generating derivatives of an isolated HSP70 molecule, comprising PEGylating the isolated HSP70 molecule using reductive amination.

3. A method of generating derivatives of an isolated HSP70 molecule, comprising PEGylating the isolated HSP70 molecule using a reaction other than reductive amination.

4. A composition comprising a PEGylated HSP 70 molecule, wherein between one and ten PEG groups are attached to the molecule.

5. A method of generating a fusion of HSP 70 with the Fc fragment of an antibody.

6. A composition comprising a recombinant HSP 70:Fc fragment fusion protein comprising a leader peptide, HSP 70, a peptide linker, and a human IgG Fc variant, wherein the variant is comprised of a hinge, CH2, and CH3 domains of human IgG1.

7. The composition of claim 6, wherein the peptide linker comprises 20 or fewer amino acids and is present between the HSP 70 and the human IgG Fc variant; and wherein the peptide linker comprises two or more amino acids selected from the group consisting of glycine, serine, alanine, and threonine.

8. A composition comprising a CHO cell line transfected with DNA encoding the fusion protein of claim 6, wherein the fusion protein is expressed in its growth medium in excess of 10 μg per million cells in a 24 hour period.

9. A composition comprising a recombinant fusion protein comprised of HSP 70, a flexible peptide linker, and a human IgG Fc variant, wherein the variant comprises a hinge, CH2, and CH3 domains of human IgG1, wherein the fusion protein is generated by the method comprising the steps of:
(a) generating a CHO cell line transfected with DNA encoding the recombinant fusion protein;
(b) growing the cell line under conditions wherein the recombinant fusion protein is expressed in its growth medium in excess of 10 μg per million cells in a 24 hour period; and
(c) purifying the expressed protein from step (b).

10. The composition of claim 9, wherein the flexible peptide linker contains 20 or fewer amino acids and is present between the HSP 70 and the human IgG Fc variant; and wherein the peptide linker comprises two or more amino acids selected from the group consisting of glycine, serine, alanine, and threonine.

11. A method of enhancing the survivability of a cell, tissue, or subject under stress, wherein the method comprises contacting exogenous HSP 70 derivatives with the cell, tissue, or subject in an amount effective to enhance the survivability of the cell, tissues, or subject.

12. The method of claim 11, wherein the cell is part of a primary neuron culture.

13. The method of claim 11, wherein the cell is part of a primary motoneuron culture.

14. The method of claim 11, wherein the subject is an animal model for a human disease state or process.

15. The method of claim 14, wherein the state or process is selected from the group consisting of cerebral ischaemia, cystic fibrosis, myocardial infarction, inflammatory disorders, hepatotoxicity, sepsis, organ or tissue transplant rejection, tumorous diseases, gastric mucosal damage, brain hemorrhage, diabetic neuropathy, diabetic retinopathy, chronic wound healing associated with diabetes and other disorders, neurodegenerative diseases, amyotrophic lateral sclerosis, Parkinson's disease, frontotemporal lobar degeneration, epilepsy, post-traumatic neuronal damage, acute renal failure, glaucoma, skin degeneration, and celiac disorder.

16. The method of claim 11, wherein the subject is a human patient.

17. The method of claim 16, wherein the patient was diagnosed with a state or process selected from the group consisting of cerebral ischaemia, cystic fibrosis, myocardial infarction, inflammatory disorders, hepatotoxicity, sepsis, organ or tissue transplant rejection, tumorous diseases, gastric mucosal damage, brain hemorrhage, diabetic neuropathy, diabetic retinopathy, chronic wound healing associated with diabetes and other disorders, neurodegenerative diseases, amyotrophic lateral sclerosis, Parkinson's disease, frontotemporal lobar degeneration, epilepsy, post-traumatic neuronal damage, acute renal failure, glaucoma, skin degeneration, and celiac disorder.
